(19) United States
(12) Patent Application Publication
(10) Pub. No.: US 2010/0015655 A1
(43) Pub. Date: Jan. 21, 2010

(54) Methods and Kits for Detecting and Quantifying Damage Caused by a Parasite

(75) Inventors: Maciej Adamczyk, Gurnee, IL (US); Roy Jeffrey Brashear, Mundelein, IL (US); Philip G. Mattingly, Third Lake, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/174,887

(22) Filed: Jul. 17, 2008

(51) Int. Cl. C12Q 1/02; G01T 1/10

(52) U.S. Cl. 435/29; 436/111; 436/104; 250/361 C

(57) ABSTRACT

The present disclosure relates to assays and kits for detecting and quantifying damage caused by a parasite in a subject and monitoring the progression of parasitic disease in a subject.

### RELATED APPLICATION INFORMATION

None.

### TECHNICAL FIELD

The present disclosure relates to assays and kits for detecting and quantifying damage caused by a parasite in a subject. Additionally, the present disclosure further relates to assays and kits for monitoring parasitic disease in a subject.

### BACKGROUND

Trypanosomiasis, malaria, Leishmaniasis and trichomoniasis are major parasitic diseases in developing countries (See, McKerrow, J. H. et al., Annu. Rev. Microbiol. 47:821-853 (1993)). For example, American trypanosomiasis or Chagas' disease, is the leading cause of heart disease in Latin America (Libow, L. F. et al., Cutis, 48:37-40 (1991)). At least 16-18 million people are infected with Trypanosoma cruzi (T. cruzi), resulting in more than 50,000 deaths each year (See, Godal, T. et al., J. Tropical diseases. WHO Division of Control in Tropical Diseases World Health Organization: Geneva, Switzerland, pp 12-13. (1990)). The statistics for malaria even more significant, with about 300-500 million clinical cases and about 3 million deaths each year. Additionally, at least 10 million people are infected with a form of Leishmania each year (See, Goodman & Gilman's The Pharmacological Basis of Therapeutics, 9th Ed, 1996, McGraw-Hill, New York).

Chagas is transmitted to humans by blood-sucking triatomine vectors with an infectious trypomastigote form of the protozoan parasite T. cruzi (See, Bonaldo, M. C. et al., Exp. Parasitol, 73:44-51 (1981); Harth, G., et al., Mol. Biochem. Parasitol, 58:17-24 (1993); Meirelles, M. N. L., et al., Mol. Biochem. Parasitol, 52:175-184 (1992)). African trypanosomiasis is transmitted to humans and cattle by tsetse flies and is caused by subspecies of T. brucei. “African sleeping sickness” is transmitted by an infectious trypomastigote from T. brucei gambiense, and T. brucei rhodesiense produces a progressive and usually fatal form of disease marked by early involvement of the central nervous system. T. brucei is further the cause of nagana in cattle, but bovine trypanosomiasis is also transmitted by T. congolense and T. evansi. In trypanosomiasis infections, the trypomastigote enters the host bloodstream and ultimately invades a cardiac muscle cell, where it transforms into the intracellular amastigote. The parasite may also be found in the blood, lymph, spinal fluid and cells of the gastrointestinal tract. Amastigotes replicate within cells, transform back to trypomastigotes, and rupture the cell, releasing the infectious form back into the bloodstream and other cells, amplifying the infection.

Malaria is caused by protozoa of the genus Plasmodium and is transmitted to humans through the bite of an infected anopheline mosquito. The parasites develop into tissue schizonts in hepatic parenchymal cells, and then are released into the circulation as merozoites, which invade erythrocytes. In erythrocytes, the merozoites mature from trophozoites into schizonts. Schizont-containing erythrocytes rupture to release merozoites that then invade more erythrocytes to continue the malarial cycle. Of the many different types of Plasmodium, only four types can infect humans, specifically, Plasmodium vivax, Plasmodium malariae, Plasmodium falciparum and Plasmodium ovale. The most serious forms of the disease in humans are caused by Plasmodium vivax and Plasmodium falciparum.

Leishmaniasis is caused by protozoal species and subspecies of Leishmania transmitted to humans by the bites of infected female phlebotamine sandflies. Promastigotes injected into the host are phagocytized by tissue monocytes and are transformed into amastigotes, which reside in intracellular phagolysosomes. Human Leishmaniasis is classified into cutaneous, diffuse cutaneous, mucocutaneous and visceral (kala azar) forms.

Trichomoniasis is a common sexually transmitted disease (STD) that affects 2 to 3 million Americans yearly. Trichomoniasis is caused by the single-celled protozoan parasite, Trichomonas vaginalis. Trichomoniasis is primarily an infection of the urogenital tract. The urethra and prostate is the most common site of infection in men, and the vagina is the most common site of infection in women.

Many significant issues result from the above parasitic diseases. Specifically, not only do these parasitic diseases cause tremendous damage and even death to the subject suffering from the disease, but these diseases can also be transmitted to other so-called “innocent” subjects by blood transfusion and congenitally.

Diagnostic tests that detect host antibodies that result from infection of a subject by a parasitic disease have been developed to screen out potentially infected individuals and blood products. One problem with these tests is that while the presence of antibodies in a subject is indicative of current or past infection, these tests do not provide any information on the extent of the damage, progression or outcome of the parasitic disease in the subject. Therefore, there is a need in the art for diagnostic tests that can be used to detect the presence and quantify the amount of damage caused by a parasite in a subject as well as monitor the damage caused by parasitic disease in a subject.

### SUMMARY

In one embodiment, the present disclosure relates to a method for detecting damage caused by a parasite in a subject. The method comprises the steps of:

a) determining the concentration of at least one biomarker in a test sample obtained from a subject, wherein the at least one biomarker is selected from the group consisting of: choline, phosphatidylcholine, lysophosphatidyl choline and phosphocholine; and

b) comparing the concentration of the at least one biomarker determined in step (a) with a predetermined level, wherein if the concentration of the biomarker determined in step (a) is lower then the predetermined level, then the subject is determined not to have experienced damage caused by a parasite and further wherein, if the concentration of the biomarker determined in step (a) is higher then the predetermined level, then the subject is determined to have experienced damage caused by a parasite.

In the above method, the parasite is selected from the group consisting of: Trypanosoma, Plasmodium, Leishmania, and Trichomonas. Additionally, in the above method, the test sample is whole blood, red blood cells, serum or plasma.

Optionally, prior to making the comparison in step (b), the above-described method further comprises the steps of:

adding at least one analyte specific enzyme to the test sample;

adding at least one chemiluminescent compound to the test sample; and

measuring the light generated from the light signal and detecting the presence of at least one biomarker present in the test sample.

The chemiluminescent compound used in the above-described method can be an acridinium compound. If an acridinium compound is added, then the above-described method can further comprise the step of adding at least one basic solution to the test sample.

Any acridinium compound can be used in the above-described method. For example, the acridinium compound can be an acridinium-9-carboxamide having a structure according to formula I:

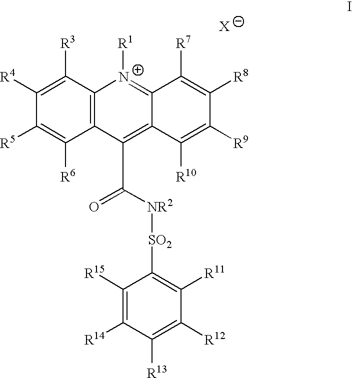

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Alternatively, the acridinium compound can be an acridinium-9-carboxylate aryl ester having a structure according to formula II:

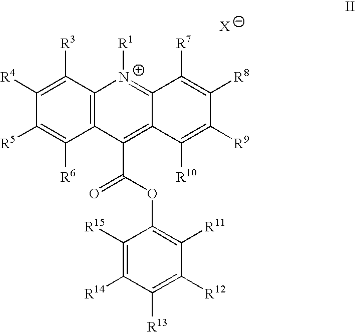

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

In another embodiment, the present invention relates to a method for quantifying damage caused by a parasite in a subject. The method comprises the steps of:

a) adding at least one analyte specific enzyme to a test sample obtained from a subject;

b) adding at least one chemiluminescent compound to the test sample;

c) measuring the light generated from the chemiluminescent compound and determining the amount of at least one biomarker present in the test sample, wherein the biomarker is selected from the group consisting of: choline, phosphatidylcholine, lysophosphatidyl choline and phosphocholine; and

d) comparing the amount of the at least one biomarker determined in step (c) with a predetermined level, wherein if the amount of the biomarker determined in step (c) is lower than the predetermined level, then the subject is determined to have a reduced severity of damage by the parasite and further wherein, if the amount of the biomarker determined in step (c) is higher then the predetermined level, then the subject is determined to have an increased severity of damage caused by the parasite.

In the above method, the parasite is selected from the group consisting of: Trypanosoma, Plasmodium, Leishmania, and Trichomonas. Additionally, in the above method, the test sample is whole blood, red blood cells, serum or plasma.

The chemiluminescent compound used in the above-described method can be an acridinium compound. If an acridinium compound is added, then the above-described method can further comprise the step of adding at least one basic solution to the test sample.

Any acridinium compound can be used in the above-described method. For example, the acridinium compound can be an acridinium-9-carboxamide having a structure according to formula I:

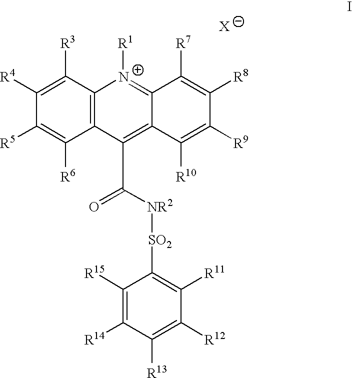

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Alternatively, the acridinium compound can be an acridinium-9-carboxylate aryl ester having a structure according to formula II:

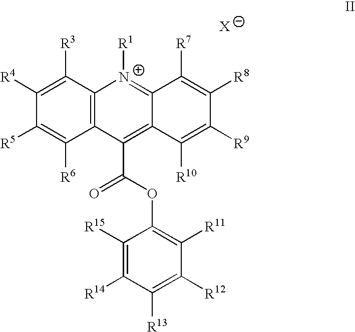

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

In yet another embodiment, the present disclosure relates to a method for monitoring progression of damage caused by parasitic disease in a subject. The method comprises the steps of:

a) determining the concentration at least one biomarker in a test sample obtained from a subject, wherein the at least one biomarker is selected from the group consisting of: choline, phosphatidylcholine, lysophosphatidyl choline and phosphocholine; and

b) comparing the concentration of the at least one biomarker determined in step (a) with a predetermined level, wherein if the concentration of the biomarker determined in step (a) is lower than the predetermined level, then the damage caused by the parasitic disease in the subject is determined not to have progressed or to have improved and further wherein if the concentration of the biomarker determined in step (a) is higher then the predetermined level, then the damage caused by the parasitic disease in the subject is determined not to have progressed.

In the above method, the parasite is selected from the group consisting of: Trypanosoma, Plasmodium, Leishmania, and Trichomonas. Additionally, in the above method, the test sample is whole blood, red blood cells, serum or plasma.

Optionally, prior to making the comparison in step (b), the above-described method further comprises the steps of:

adding at least one analyte specific enzyme to the test sample;

adding at least one chemiluminescent compound to the test sample; and

measuring the light generated from the light signal and detecting the presence of at least one biomarker present in the test sample.

The chemiluminescent compound used in the above-described method can be an acridinium compound. If an acridinium compound is added, then the above-described method can further comprise the step of adding at least one basic solution to the test sample.

Any acridinium compound can be used in the above-described method. For example, the acridinium compound can be an acridinium-9-carboxamide having a structure according to formula I:

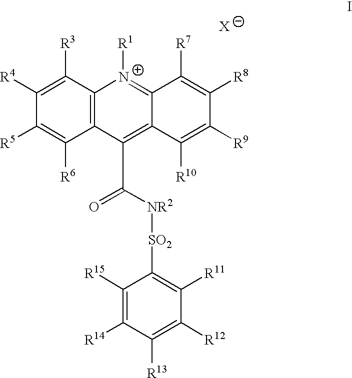

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Alternatively, the acridinium compound can be an acridinium-9-carboxylate aryl ester having a structure according to formula II:

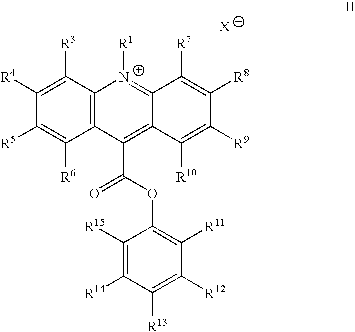

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

In yet another embodiment, the present disclosure relates to a kit for use in determining the presence or amount of damage caused by a parasite in a subject. The kit comprises:
a. at least one analyte specific enzyme;
b. at least one chemiluminescent compound; and
c. instructions for detecting the presence or amount of damage caused by a parasite in a test sample.

In the above kit, the chemiluminescent compound can be an acridinium compound. If the kit contains an acridinium compound, the kit can also further contain at least one basic solution. Any acridinium compound can be used in the above described kit. For example, the acridinium compound can be an acridinium-9-carboxamide having a structure according to formula I:

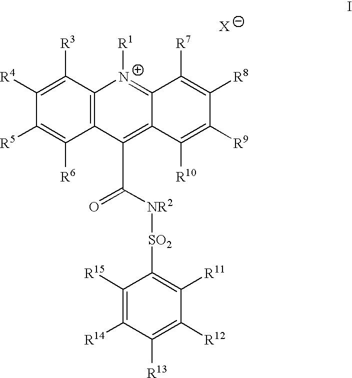

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Alternatively, the acridinium compound can be an acridinium-9-carboxylate aryl ester having a structure according to formula II:

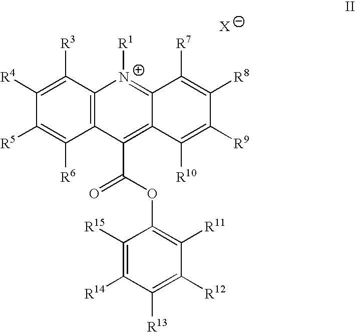

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

In the above kit, the parasite is selected from the group consisting of: Trypanosoma, Plasmodium, Leishmania, and Trichomonas.

In yet another embodiment, the present disclosure relates to a kit for monitoring parasitic disease in a subject. The kit comprises:
a. at least one analyte specific enzyme;
b. at least one chemiluminescent compound; and
c. instructions for monitoring parasitic disease in an individual.

In the above kit, the chemiluminescent compound can an acridinium compound. If the kit contains an acridinium compound, the kit can also further contain at least one basic solution. Any acridinium compound can be used in the above described kit. For example, the acridinium compound can be an acridinium-9-carboxamide having a structure according to formula I:

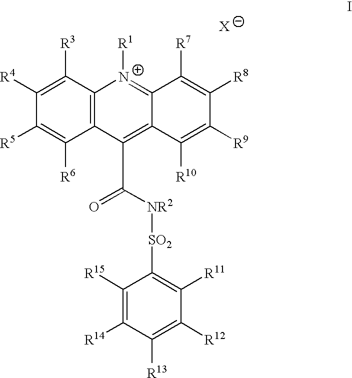

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Alternatively, the acridinium compound can be an acridinium-9-carboxylate aryl ester having a structure according to formula II:

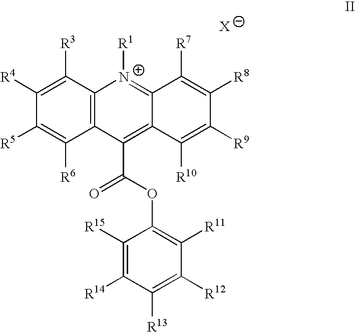

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

In the above kit, the parasite is selected from the group consisting of: Trypanosoma, Plasmodium, Leishmania, and Trichomonas.

### BRIEF DESCRIPTION OF THE FIGURE

FIG. 1 shows a distribution plot of the level of free choline measured in a population that tested positive for Chagas disease compared to a population of healthy normal blood donors.

### DETAILED DESCRIPTION

The present disclosure relates to methods for detecting and quantifying damage caused by at least one parasite in a subject. Additionally, the methods of the present disclosure can also be used to monitor the progression of damage caused by parasitic disease in a subject. Specifically, the inventors have found that detecting the presence of and quantifying the amount of at least one biomarker of cell-membrane degradation in a test sample can be used to detect and quantify the amount of damage caused by a parasite as well as monitor the progression of damage caused by parasitic disease in a subject.

### A. Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Acyl (and Other Chemical Structural Group Definitions)

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present disclosure can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂.

As used herein, the term “carboxyalkyl” refers to a —(CH₂)ₙCO₂H or —(CH₂)ₙCO₂⁻ group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Representative examples of cycloalkenyl groups include cyclohexenyl, octahydronaphthalenyl, norbomylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present disclosure can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

b) Analyte Specific Enzyme

As used herein, the term “analyte specific enzyme” refers to an enzyme that is capable of generating hydrogen peroxide. Examples of analyte specific enzymes are listed below in Table 1.

TABLE 1

| | IUBMB ENZYME | PREFERRED |
|---|---|---|
| ACCEPTED COMMON NAME | NOMENCLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy-2-(4- |
| | | hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| Aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol |
| | | (2-naphthyl)methanol |
| | | 3-methoxybenzyl alcohol |
| aryl-aldehyde oxidase | EC 1.2.3.9 | an aromatic aldehyde |
| Catechol oxidase | EC 1.1.3.14 | Catechol |
| cholesterol oxidase | EC 1.1.3.6 | Cholesterol |
| Choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| Ecdysone oxidase | EC 1.1.3.16 | Ecdysone |
| ethanolamine oxidase | EC 1.4.3.8 | Ethanolamine |
| Galactose oxidase | EC 1.1.3.9 | D-galactose |
| Glucose oxidase | EC 1.1.3.4 | β-D-glucose |
| glutathione oxidase | EC 1.8.3.3 | Glutathione |
| Glycerol-3-phosphate oxidase | EC 1.1.3.21 | sn-glycerol 3-phosphate |
| Glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| Oxalate oxidase | EC 1.2.3.4 | Oxalate |
| polyamine oxidase | EC 1.5.3.11 | 1-N-acetylspermine |
| polyphenol oxidase | EC 1.14.18.1 | |
| Polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| Pyranose oxidase | EC 1.1.3.10 | D-glucose |
| | | D-xylose |
| | | L-sorbose |
| | | D-glucono-1,5-lactone |
| Pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| Pyruvate oxidase | EC 1.2.3.3 | Pyruvate |
| Pyruvate oxidase (CoA-acetylating) | EC 1.2.3.6 | Pyruvate |
| Reticuline oxidase | EC 1.21.3.3 | Reticuline |
| retinal oxidase | EC 1.2.3.11 | Retinal |
| Rifamycin-B oxidase | EC 1.10.3.6 | rifamycin-B |
| Sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| Thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| Vanillyl-alcohol oxidase | EC 1.1.3.38 | vanillyl alcohol |
| Xanthine oxidase | EC 1.17.3.2 | Xanthine |
| xylitol oxidase | EC 1.1.3.41 | Xylitol |

c) Anion

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

d) Biomarker-Specific Enzyme

As used herein, the phrase “biomarker-specific enzyme” refers to an enzyme that is capable of producing free choline from choline esterified to one or more phospholipids (“PL-choline”). Examples of biomarker-specific enzymes include, but are not limited to, phospholipases and lysophospholipases.

e) Free Choline

As used herein, the phrase “free choline” refers to choline that is not esterified to one or more phosopholipids and has a formula of formula III:

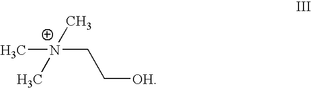

While free choline is structurally represented above formula III, in the absence of a counterion, one of ordinary skill in the art would understand that a counterion can be present. One of ordinary skill in the art would also understand that any free choline present in a test sample can comprise a mixture of two or more counterions. The presence/absence of a counterion does not materially affect the method of any assay.

f) Predetermined Level

As used herein, the term “predetermined level” refers generally at an assay cutoff value that is used to assess diagnostic results by comparing the assay results against the predetermined level, and where the predetermined level already that has been linked or associated with various clinical parameters (e.g., assessing risk, severity of disease, progression/nonprogression/improvement, etc.). The present disclosure provides exemplary predetermined levels, and describes the initial linkage or association of such levels with clinical parameters for exemplary immunoassays as described herein. However, it is well known that cutoff values may vary dependent on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this description.

g) Specific Binding Partner

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

h) Subject

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). Preferably, the subject is a human. Most preferably, the subject is a human that is suspected of having been infected by a parasite or suspected of having parasitic disease or that has actually been infected by a parasite or is actually known to be suffering from parasitic disease.

i) Test Sample

As used herein, the term “test sample” generally refers to a biological material being tested for and/or suspected of containing at least one biomarker of cell membrane degradation and that is obtained from a subject. Preferably, the at least one biomarker is selected from the group consisting of: choline, phosphatidylcholine, lysophosphatidyl choline, phosphocholine and any combinations thereof. The test sample may be derived from any biological source, such as, a physiological fluid, including, but not limited to, whole blood, red blood cells, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the biomarker.

### B. Assay for Detecting or Quantifying the Damage Caused by a Parasite in a Subject and Monitoring the Progression of Parasitic Disease in a Subject

The present disclosure relates to assays for detecting and quantifying damage caused by at least one parasite in a subject as well as monitoring the progression of damage caused by parasitic disease in a subject. Examples of parasites include, but are not limited to, Trypanosoma (e.g., Trypanosoma cruzi, Trypanosoma brucei, Trypanosoma congolense, Trypanosoma evansi), Plasmodium (Plasmodium vivax, Plasmodium malariae, Plasmodium falciparum, Plasmodium ovale), Leishmania (e.g., visceral Leishmaniasis, cutaneous Leishmaniasis, mucocutaneous Leishmaniasis, diffuse cutaneous Leishmaniasis), Trichomonas (e.g., Trichomonas vaginalis), etc. Parasites are known to cause parasitic disease in a subject. Examples of parasitic disease include, but are not limited to, Chagas, African sleeping sickness, nagana, malaria, Leishmaniasis, sexually transmitted disease (STD), etc.

The assay or method of the present disclosure involves obtaining a test sample from a subject and then detecting the presence of or quantifying the amount of at least one biomarker of cell membrane degradation in the test sample. Preferably, the at least one biomarker of cell membrane degradation is a biomarker selected from the group consisting of: choline, phosphatidylcholine, lysophosphatidyl choline, phosphocholine and any combinations thereof. Specifically, the present disclosure relates to detecting and quantifying the amount of at least one biomarker of cell-membrane degradation in a test sample. The presence of and the concentration (e.g., amount) of at least one biomarker in a test sample can be used to detect and quantify the damage caused by a parasite in a subject as well as monitor the progression of the damage caused by parasitic disease in a subject.

In one embodiment of the present disclosure, a test sample is obtained from the subject using routine techniques known to those skilled in the art. The test sample contains, is suspected of containing or is believed to contain at least one biomarker of cell membrane degradation. Optionally, in one aspect of the present disclosure, at least one biomarker-specific enzyme is added to the test sample. The at least one biomarker-specific enzyme can be added to the test sample to produce free choline. The time at which the at least one biomarker-specific enzyme is added to the test sample is not critical provided that it is added before determining the concentration of the at least one biomarker in the test sample. The concentration of the at least one biomarker in the test sample is then determined using routine techniques in the art.

Specifically, at least one analyte-specific enzyme is added to the test sample in order to generate peroxide. The amount of at least one analyte-specific enzyme that can be added to the test sample is from about 0.0001 unit/mL to about 10,000 units/mL. Preferably, the at least one analyte-specific enzyme is at least one oxidase. More preferably, the at least one analyte-specific enzyme is choline oxidase. The time at which the at least one analyte-specific enzyme is added to the test sample is not critical provided that it is added before the peroxide is converted to an end product having a distinct chemiluminescent emission, namely, before the addition of at least one chemiluminescent compound as will be further discussed herein.

The peroxide generated in the test sample by the addition of one or more analyte-specific enzymes can be detected electrochemically or can be converted to an end product having a distinct color or absorbance wavelength in the ultraviolet or visible range, or a fluorescent or chemiluminescent emission. Preferably, peroxide generated in the test sample can be converted to an end product having a distinct chemiluminescent emission. Such an end product is produced by adding to the test sample at least one at least one chemiluminescent compound. Any chemiluminescent compound can be added to the test sample. Examples of chemiluminescent compounds that can be used include, but are not limited to, acridinium esters, thioesters, sulfonamides; luminol, isoluminol, phenanthridinium esters, and the like. Preferably, the chemiluminescent compound is at least one acridinium compound. More preferably, the acridinium compound is an acridinium carboxamide. An example of an acridinium carboxamide that can be used is an acridinium-9-carboxamide having a structure according to formula I:

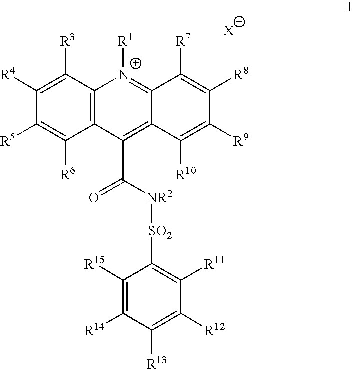

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium carboxamide is an acridinium-9-carboxylate aryl ester. The acridinium-9-carboxylate aryl ester having a structure according to formula II:

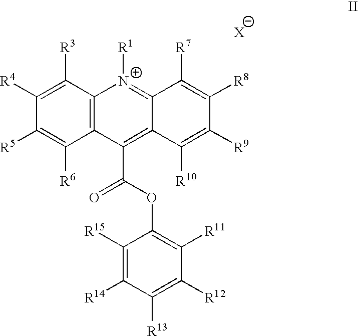

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

Examples of acridinium-9-carboxylate aryl esters having the above formula II that can be used in the present disclosure include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

After the addition of the acridinium compound to the test sample, at least one basic solution is added to the test sample in order to generate a detectable signal, namely, a chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the processed test sample depends on the concentration of the basic solution used in the method. Based on the concentration of the basic solution used, one skilled in the art could easily determine the amount of basic solution to be used in said method.

The chemiluminescent signal that is generated can then be detected using routine techniques known to those skilled in the art. Thus, in the assays of the present disclosure, the chemiluminescent signal generated after the addition of a basic solution, indicates the presence of at least one biomarker. The amount of the at least one biomarker in the test sample can be quantified based on the intensity of the signal generated. Specifically, the amount of the at least one biomarker contained in a test sample is either proportional or inversely proportional to the intensity of the signal generated. For example, in some instances, a high signal intensity may be generated by the lowest concentration of biomarker in the test sample (in this instance, the amount of the at least one biomarker in the test sample is inversely proportional to the amount of signal generated). Specifically, the amount of said at least one biomarker present can be quantified based on comparing the amount of light generated to a standard curve for the biomarker or by comparison to a reference standard. The standard curve can be generated using serial dilutions or solutions of at least one biomarker of known concentration, by mass spectroscopy, gravimetrically and by other techniques known in the art.

As mentioned previously herein, once the concentration of the at least one biomarker is determined, then the concentration of said at least one biomarker is compared with a predetermined level. In one aspect, if the concentration of the biomarker in the test sample is lower then the predetermined level, then the subject is determined not to have experienced damage caused by a parasite. If the concentration of the biomarker in the test sample is higher then the predetermined level, then the subject is determined to have experienced damage caused by the parasite.

In another aspect, when quantifying the amount of damage caused by a parasite in a subject if the amount of the biomarker determined in a test sample is lower than the predetermined level, then the subject is determined to have a reduced severity of damage by the parasite. If the amount of the biomarker determined in the test sample is higher then the predetermined level, then the subject is determined to have an increased severity of damage caused by the parasite.

In yet another aspect, when monitoring for the progression of damage caused by parasitic disease in a subject, if the concentration of the biomarker in the test sample is lower than the predetermined level, then the damage caused by the parasitic disease in the subject is determined not to have progressed or to have improved. If the concentration of the biomarker determined in the test sample is higher then the predetermined level, then the damage caused by the parasitic disease in the subject is determined not to have progressed.

In second embodiment of the present disclosure, a test sample is obtained from the subject using routine techniques known to those skilled in the art as discussed previously herein. The test sample is treated to remove or separate the at least one biomarker from any other proteins contained in the test sample. The at least one biomarker can be removed or separated from the test sample using immuno-separation techniques that are well known to those skilled in the art. Specifically, in such techniques, the test sample suspected of containing at least one biomarker is contacted with a specific binding partner that binds to the at least one biomarker thus forming an analyte-specific binding partner complex. In one aspect, the specific binding partner can be used in a sandwich type format or a competitive format, the techniques for which are well known in the art. An example of a specific binding partner that can be used is an antibody, namely, an antibody that binds to the at least one biomarker. For example, if the at least one biomarker is choline, then the specific binding partner is an antibody that is capable of binding to choline. The analyte specific binding partner complex is then removed or separated from the test sample using routine techniques known in the art, such as, but not limited to, washing, thus resulting in an analyte specific binding partner complex sample.

The specific binding partner used to remove or separate the at least one biomarker can be immobilized on a solid phase. The solid phase can be any material known to those of ordinary skill in the art to which the specific binding partners, such as, but not limited to, antibodies or antigens, can be attached. Examples of solid phases that can be used, include, but are not limited to, a test well in a microtiter plate, nitrocellulose, nylon, a bead or a disc (which can be made out of glass, fiberglass, latex, plastic or a paper material), a gel (for example, a gel through which the polypeptides have been run and which is subsequently dried) or a strip, disc or sheet (which can be made out of nitrocellulose, nylon, plastic or paper). The specific binding partner can be bound to the solid phase by adsorption, by covalent bonding using a chemical coupling agent or by other means known in the art, provided that such binding does not interfere with the ability of the specific binding partner to bind to the at least one biomarker. Moreover, if necessary, the solid phase can be derivatized to allow reactivity with various functional groups on any of the specific binding partner. Such derivatization requires the use of certain coupling agents such as, but not limited to, maleic anhydride, N-hydroxysuccinimide and 1-ethyl-3-(3-dimethylaminopropyl)carbodiimide.

Optionally, in another aspect of the present disclosure, if at least one of the biomarkers in the at least one analyte-specific binding partner complex sample is esterified choline, at least one biomarker-specific enzyme can be added to the analyte-specific binding partner complex sample to produced free choline. The time at which the at least one biomarker-specific enzyme is added to the analyte-specific binding partner complex sample is not critical provided that it is added before determining the concentration of the at least one biomarker. The concentration of the at least one biomarker is then determined using routine techniques in the art.

Specifically, at least one analyte-specific enzyme is added to the at least one analyte-specific binding partner complex sample to generate peroxide in the sample. The amount of at least analyte-specific enzyme that can be added to the analyte specific binding partner complex sample is from about 0.0001 unit/mL to about 10,000 units/mL. The time at which the at least one analyte-specific enzyme is added to the at least one analyte specific binding partner complex sample is not critical, provided that it is added before the peroxide is converted to an end product having a distinct chemiluminescent emission, namely, before the addition of at least one chemiluminescent compound as discussed previously herein.

Optionally, after the generation of the peroxide, the same specific binding partner (namely, the first specific binding partner) used to remove or separate the at least one biomarker from the test sample, or a second specific binding partner (which is different from the first specific binding partner), can be used to remove the analyte specific binding partner complex from the analyte specific binding partner complex sample (thus leaving just the peroxide in the sample).

As discussed previously herein, the peroxide that is generated by the addition of the at least one analyte-specific enzyme can then be converted to an end product having a distinct chemiluminescent emission. Such an end product is produced by adding to the analyte-specific binding partner complex sample at least one chemiluminescent compound. Any chemiluminescent compound can be added to the test sample. Examples of chemiluminescent compounds that can be used include, but are not limited to, acridinium esters, thioesters, sulfonamides; luminol, isoluminol, phenanthridinium esters, and the like. Preferably, the at least one chemiluminescent compound is at least one acridinium compound. Most preferably, the at least one acridinium compound is an acridinium carboxamide (i.e., the acridinium carboxamide having the formula of formula I or formula II) which was previously discussed herein.

After the addition of the acridinium compound to the analyte specific binding partner complex sample (which may or may not still contain the analyte specific binding partner complex), at least one basic solution is added in order to generate a detectable signal, namely, a chemiluminescent signal. The basic solution is the same basic solution discussed previously herein, namely, a solution that contains at least one base and that has a pH greater than or equal to 10, preferably, greater than or equal to 12. As also discussed previously herein, the chemiluminescent signal generated can be detected using routine techniques known to those skilled in the art.

### C. Assay Kits

In another embodiment, the present disclosure relates to a kit for determining the presence or amount of a parasite in a subject or monitoring the progression parasitic disease in a subject. In one aspect, the kit can contain at least one chemiluminescent compound. Preferably, the at least one chemiluminescent compound is at least one acridinium compound. The acridinium compound may comprise at least one acridinium-9-carboxamide, at least one acridinium-9-carboxylate aryl ester or any combinations thereof. More specifically, the acridinium-9-carboxamide that can be used has the structure according to Formula I:

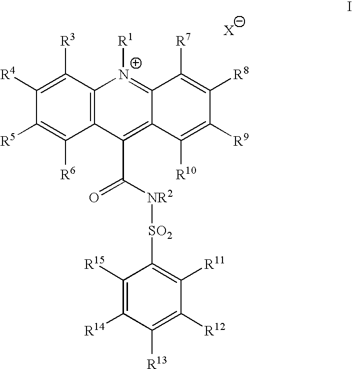

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present, X^{⊖}is an anion.

Additionally, the acridinium-9-carboxylate aryl ester that can be used has a structure according to formula II:

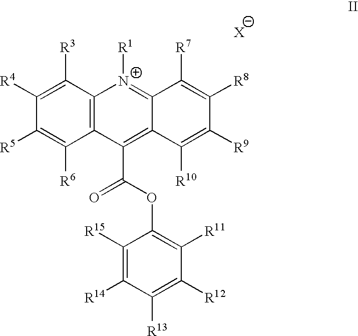

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖}is an anion.

Also, the kit can also contain one or more instructions for detecting and quantifying at least one biomarker in a test sample for the purposes of detecting or quantifying the amount of damage caused by a parasite or for monitoring parasitic disease in a subject. The kit can also contain instructions for generating a standard curve for the purposes of quantifying the amount of the at least one biomarker in the test sample or a reference standard for purposes detecting or quantifying the amount of damage caused by a parasite or for monitoring parasitic disease in an individual.

Optionally, the kit may also contain at least one analyte-specific enzyme, such as at least one enzyme listed in Table 1.

Additionally, the kit may also contain at least one specific binding partner. Optionally, the at least one specific binding partner contained in the kit can have conjugated thereon at least one analyte-specific enzyme. The at least one analyte-specific enzyme can be at least one enzyme listed in Table 1. If the at least one specific binding partner included in the kit has conjugated thereon at least one analyte-specific enzyme, then the kit can further contain at least one substrate. The at least one substrate that can be included in the kit includes, but is not limited to, substrates listed in Table 1.

Optionally, the kit can also contain at least one biomarker-specific enzyme.

### D. Adaptations of the Methods of the Present Disclosure

The disclosure as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II instruments, as well as other platforms. Moreover, the disclosure optionally is adaptable for the Abbott Laboratories commercial Point of Care (i-STAT™) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

By way of example, and not of limitation, examples of the present disclosure shall now be given.

### EXAMPLE 1

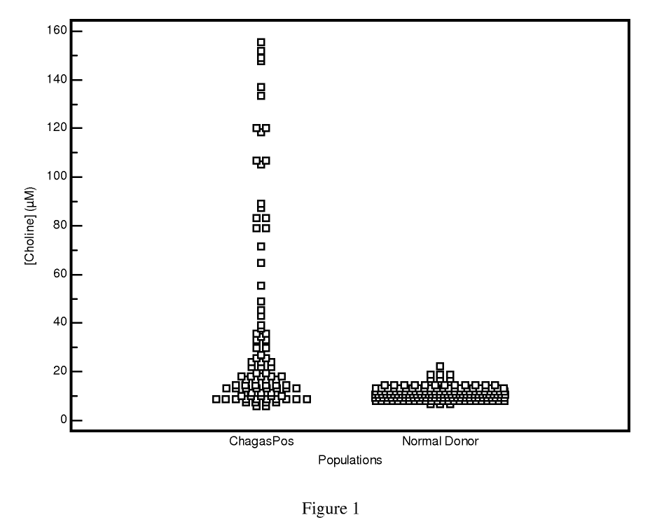

A series of plasma and serum samples (n=91) that tested positive for Chagas disease were analyzed for choline using the method of Adamczyk, M., Brashear, R. J., Mattingly, P. G., and Tsatsos, P. H., “Homogeneous chemiluminescent assays for free choline in human plasma and whole blood,” Anal. Chim. Acta, 579:61-67 (2006)). The results were compared to the level of choline found in a normal blood donor population (n=161) (See, FIG. 1). Statistical analysis of the two populations using the Mann-Whitney test for independent samples (See, Table 2, below), indicated that the choline concentration is significantly elevated in the population that had tested positive for Chagas disease compared to that in the normal donor population.

TABLE 2

| | Chagas Positive | Normal_Donor |
|---|---|---|
| Sample size (n) | 91 | 161 |
| Lowest value | 5.8700 | 6.7000 |
| Highest value | 155.4300 | 22.5000 |
| Median | 19.0600 | 11.2800 |
| 95% Cl for the median | 15.2133 to 25.8145 | 10.9000 to 11.8966 |
| Interquartile range | 12.0475 to 44.6800 | 9.7300 to 13.1325 |

| | |
|---|---|
| Average rank of Chagas | 170.2363 |
| Positive group | |
| Average rank of Normal | 101.7795 |
| Donor group | |
| Large sample test statistic Z | 7.161101 |
| Two-tailed probability | P < 0.0001 |

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the disclosure. It will be readily apparent to one skilled in the art that varying substitutions and modifications may be made to the disclosure disclosed herein without departing from the scope and spirit of the disclosure.

All patents and publications mentioned in the specification are indicative of the levels of those skilled in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

What is claimed is:

1. A method for detecting damage caused by a parasite in a subject, the method comprising the steps of:
a) determining the concentration of at least one biomarker in a test sample obtained from a subject, wherein the at least one biomarker is selected from the group consisting of: choline, phosphatidylcholine, lysophosphatidyl choline and phosphocholine; and
b) comparing the concentration of the at least one biomarker determined in step (a) with a predetermined level, wherein if the concentration of the biomarker determined in step (a) is lower then the predetermined level, then the subject is determined not to have experienced damage caused by a parasite and further wherein, if the concentration of the biomarker determined in step (a) is higher then the predetermined level, then the subject is determined to have experienced damage caused by a parasite.

2. The method of claim 1, wherein the parasite is selected from the group consisting of: Trypanosoma, Plasmodium, Leishmania, and Trichomonas.

3. The method of claim 1, wherein the test sample is whole blood, red blood cells, serum or plasma.

4. The method of claim 1, wherein prior to making the comparison in step (b), the method further comprises the steps of
adding at least one analyte-specific enzyme to the test sample to generate peroxide;
adding at least one chemiluminescent compound to the test sample; and
measuring the light generated from the light signal and detecting the presence of at least one biomarker present in the test sample.

5. The method of claim 4, wherein the chemiluminescent compound is an acridinium compound.

6. The method of claim 5, wherein the method further comprises adding at least one basic solution to the test sample.

7. The method of claim 5, wherein the acridinium compound is an acridinium-9-carboxamide having a structure according to formula I: 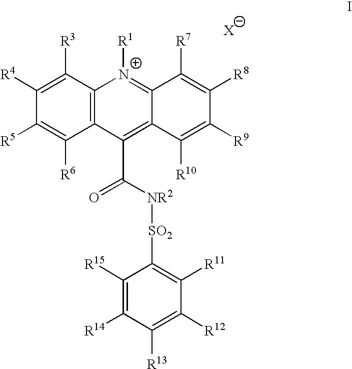
wherein R¹ and R² are each independently selected from the group consisting of: alkyl, alkenyl, alkynyl, aryl or aralkyl, sulfoalkyl, carboxyalkyl and oxoalkyl; and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and
optionally, if present, X^{⊖}is an anion.

8. The method of claim 5, wherein the acridinium compound is an acridinium-9-carboxylate aryl ester having a structure according to formula II: 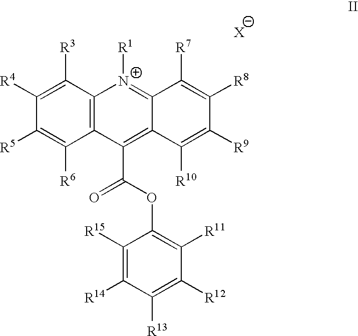
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖}is an anion.

9. A method for quantifying damage caused by a parasite in a subject, the method comprising the steps of:
a) adding at least one analyte-specific enzyme to a test sample obtained from a subject to generate peroxide;
b) adding at least one chemiluminescent compound to the test sample;
c) measuring the light generated from the chemiluminescent compound and determining the amount of at least one biomarker present in the test sample, wherein the biomarker is selected from the group consisting of: choline, phosphatidylcholine, lysophosphatidyl choline and phosphocholine; and
d) comparing the amount of the at least one biomarker determined in step (c) with a predetermined level, wherein if the amount of the biomarker determined in step (c) is lower than the predetermined level, then the subject is determined to have a reduced severity of damage by the parasite and further wherein, if the amount of the biomarker determined in step (c) is higher then the predetermined level, then the subject is determined to have an increased severity of damage caused by the parasite.

10. The method of claim 9, wherein the parasite is selected from the group consisting of: Trypanosoma, Plasmodium, Leishmania, and Trichomonas.

11. The method of claim 9, wherein the test sample is whole blood, red blood cells, serum or plasma.

12. The method of claim 9, wherein the chemiluminescent compound is an acridinium compound.

13. The method of claim 12, wherein the method further comprises adding at least one basic solution to the test sample.

14. The method of claim 12, wherein the acridinium compound is an acridinium-9-carboxamide having a structure according to formula I: 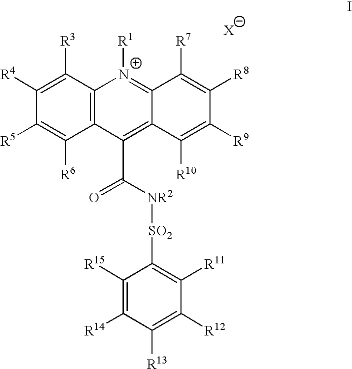
wherein R¹ and R² are each independently selected from the group consisting of: alkyl, alkenyl, alkynyl, aryl or aralkyl, sulfoalkyl, carboxyalkyl and oxoalkyl; and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and
optionally, if present, X^{⊖}is an anion.

15. The method of claim 12, wherein the acridinium compound is an acridinium-9-carboxylate aryl ester having a structure according to formula II: 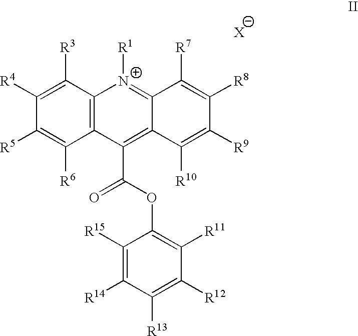
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖}is an anion.

16. A method for monitoring progression of damage caused by parasitic disease in a subject, the method comprising the steps of:
a) determining the concentration at least one biomarker in a test sample obtained from a subject, wherein the at least one biomarker is selected from the group consisting of: choline, phosphatidylcholine, lysophosphatidyl choline and phosphocholine; and
b) comparing the concentration of the at least one biomarker determined in step (a) with a predetermined level, wherein if the concentration of the biomarker determined in step (a) is lower than the predetermined level, then the damage caused by the parasitic disease in the subject is determined not to have progressed or to have improved and further wherein if the concentration of the biomarker determined in step (a) is higher then the predetermined level, then the damage caused by the parasitic disease in the subject is determined not to have progressed.

17. The method of claim 16, wherein the parasite is selected from the group consisting of: Trypanosoma, Plasmodium, Leishmania, and Trichomonas.

18. The method of claim 16, wherein the test sample is whole blood, red blood cells, serum or plasma.

19. The method of claim 16, wherein prior to making the comparison in step (b), the method further comprises the steps of
adding at least one analyte-specific enzyme to the test sample to generate peroxide;
adding at least one chemiluminescent compound to the test sample; and
measuring the light generated from the light signal and detecting the presence of at least one biomarker present in the test sample.

20. The method of claim 19, wherein the chemiluminescent compound is an acridinium compound.

21. The method of claim 20, wherein the method further comprises adding at least one basic solution to the test sample.

22. The method of claim 20, wherein the acridinium compound is an acridinium-9-carboxamide having a structure according to formula I: 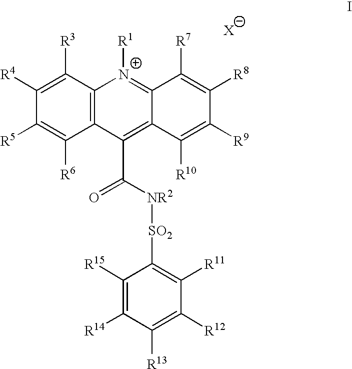
wherein R¹ and R² are each independently selected from the group consisting of: alkyl, alkenyl, alkynyl, aryl or aralkyl, sulfoalkyl, carboxyalkyl and oxoalkyl; and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and
optionally, if present, X^{⊖}is an anion.

23. The method of claim 20, wherein the acridinium compound is an acridinium-9-carboxylate aryl ester having a structure according to formula II: 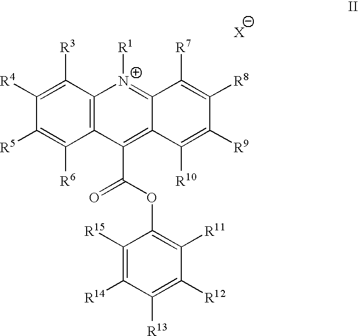
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖}is an anion.

24. A kit for use in determining the presence or amount of damage caused by a parasite in a subject, the kit comprising:
a. at least one analyte specific enzyme;
b. at least one chemiluminescent compound; and
c. instructions for detecting the presence or amount of damage caused by a parasite in a test sample.

25. The kit of claim 24, wherein the chemiluminescent compound is an acridinium compound.

26. The kit of claim 25, wherein the kit further comprises at least one basic solution.

27. The kit of claim 25, wherein the acridinium compound is an acridinium-9-carboxamide having a structure according to formula I: 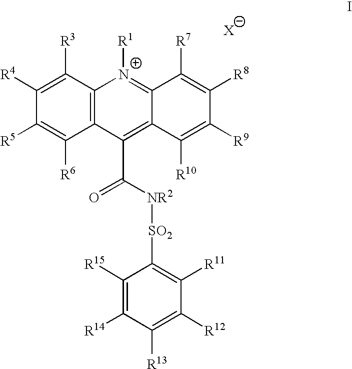
wherein R¹ and R² are each independently selected from the group consisting of: alkyl, alkenyl, alkynyl, aryl or aralkyl, sulfoalkyl, carboxyalkyl and oxoalkyl; and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and
optionally, if present, X^{⊖}is an anion.

28. The kit of claim 25, wherein the acridinium compound is an acridinium-9-carboxylate aryl ester having a structure according to formula II: 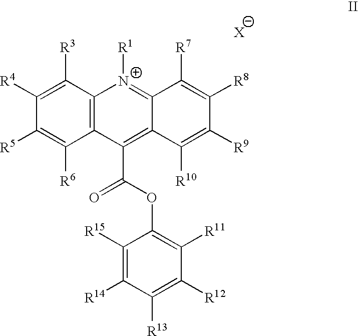
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖}is an anion.

29. The kit of claim 24, wherein the parasite is selected from the group consisting of: Trypanosoma, Plasmodium, Leishmania, and Trichomonas.

30. A kit for monitoring parasitic disease in a subject, the kit comprising:
a. at least one analyte specific enzyme;
b. at least one chemiluminescent compound; and
c. instructions for monitoring parasitic disease in an individual.

31. The kit of claim 30, wherein the chemiluminescent compound is an acridinium compound.

32. The kit of claim 31, wherein the kit further comprises at least one basic solution.

33. The kit of claim 31, wherein the acridinium compound is an acridinium-9-carboxamide having a structure according to formula I: 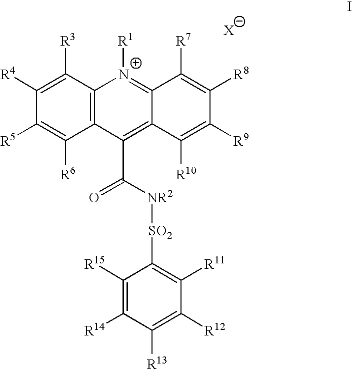
wherein R¹ and R² are each independently selected from the group consisting of: alkyl, alkenyl, alkynyl, aryl or aralkyl, sulfoalkyl, carboxyalkyl and oxoalkyl; and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and
optionally, if present, X^{⊖}is an anion.

34. The kit of claim 31, wherein the acridinium compound is an acridinium-9-carboxylate aryl ester having a structure according to formula II: 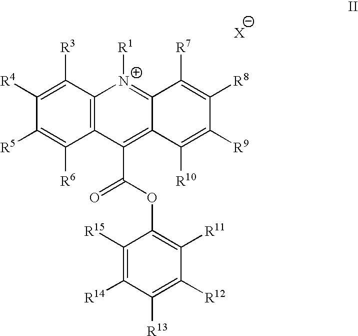
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖}is an anion.

35. The kit of claim 30, wherein the parasitic disease is selected from the group consisting of: Chagas' disease, African sleeping sickness, nagana, malaria, Leishmaniasis and sexually transmitted disease.
